(19) United States
(12) Patent Application Publication
(10) Pub. No.: US 2002/0071859 A1
(43) Pub. Date: Jun. 13, 2002

(54) Towelette product with sunscreen agent

(75) Inventors: Robert Edward Gott, Norwalk, CT (US); Craig Stephen Slavtcheff, Guilford, CT (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 09/841,208

(22) Filed: Apr. 24, 2001

Related U.S. Application Data
(60) Provisional application No. 60/242,648, filed on Oct. 23, 2000.

(51) Int. Cl. A61K 7/42; A61K 9/70

(52) U.S. Cl. 424/443; 424/59

(57) ABSTRACT

A cosmetic towelette is provided which includes a water-insoluble substrate and a fluid cosmetic composition impregnated into the substrate. The composition has a viscosity ranging from about 1 cps to 10,000 cps. The composition includes a water phase, a sunscreen phase and a surfactant system. The sunscreen phase is immiscible with the water phase and contains at least 25% organic sunscreen agent. Towelettes impregnated with the composition impart an effective SPF to the skin when applied thereto.

### BACKGROUND OF THE INVENTION

1. Field of the Invention

The invention concerns towelettes which deliver sunscreen agents to the skin in a highly efficient manner.

2. The Related Art

Sunscreen compositions are commonly used during outdoor work or leisure. They protect exposed skin against sunburn, cancer and even photo aging. Many effective sunscreen preparations are sold commercially or are described in cosmetic or pharmaceutical literature. In general, sunscreen preparations are formulated as creams, lotions or oils containing as the active agent an ultraviolet radiation absorbing chemical compound. The active agent functions by blocking passage of erythematogenic radiation thereby preventing its penetration into the skin.

Organic sunscreen agents are subject to various problems. A prime one is skin irritation. Some people are quite sensitive to organic molecules with chromophoric groups. Adverse allergic reactions can result. Therefore, it would be quite desirable to minimize the level of such compounds in sunscreen compositions. Nonetheless, minimization of the organic sunscreen level must not interfere with Sunscreen Protection Factor (SPF) activity.

Illustrative of the art is U.S. Pat. No. 5,961,961 (Dobkowski et al.) disclosing relatively viscous lotion and cream products intended for sun protection. A boost in SPF is achieved by inclusion of large particle size inorganic sunscreen agents while still formulating with relatively lower levels of organic sunscreen agent. The inorganic pigments such as titanium dioxide improved SPF while not significantly interfering with aesthetic properties.

A problem with liquid formulations is that they do not always evenly distribute over an applied surface. Another matter of concern is efficient deposition of actives. Creams and lotions do not always effectively deliver.

Applicators such as pads have been known as vehicles for cosmetic ingredients. For instance, U.S. Pat. No. 5,620,694 (Girardot) discloses a dual textured treatment pad impregnated with suitable medicated, cleansing or cosmetic compositions. Among ingredients listed for delivery are low pH actives such as salicylic acid and sunscreen agents.

None of the known art has hitherto fully solved the problem of delivering sunscreen agents evenly to the skin and in a manner that maximizes SPF value.

Accordingly, it is an object of the present invention to provide a product and method for imparting sun protection to skin surfaces.

Another object of the present invention is to provide a product and method that will evenly distribute sunscreen over an applied surface.

These and other objects of the present invention will become more apparent from the following summary and detailed discussion which follow.

### SUMMARY OF THE INVENTION

A cosmetic towelette is provided which includes:

(a) a water-insoluble substrate; and

(b) a cosmetic composition impregnated into the substrate, the composition having a viscosity ranging from about 1 to about 10,000 cps, as measured on a Brookfield LVT Viscometer using spindle 4 at 30 rpm at 25° C., the composition including:

(i) from about 80 to about 99% of a water phase;

(ii) from about 0.1 to about 20% of a sunscreen phase immiscible with the water phase, the sunscreen phase including at least 25% of an organic sunscreen agent; and

(iii) from about 0.1 to about 10% of a surfactant system to stably disperse the sunscreen phase within the water phase.

### DETAILED DESCRIPTION OF THE INVENTION

Now it has been found that towelettes impregnated with sunscreen agent can deliver this material to the skin in a highly efficient manner. Two aspects are important to achieve this effect. The first is that the cosmetic composition have a relatively thin viscosity. Secondly, the sunscreen agent concentration must be at least 25% within a sunscreen phase immiscible with a water phase of the composition.

Compositions of the present invention will have a viscosity ranging from about 1 to about 10,000 cps, preferably from about 5 to about 1,000 cps, optimally from about 5 to about 500 cps as measured with a Brookfield LVT viscometer using spindle 4 at 30 rpm as measured at 25° C. A water and a sunscreen phase constitute the cosmetic composition. The water phase may range in amount from about 80 to about 99%, preferably from about 85 to about 95%, optimally from about 90 to about 95% by weight. The sunscreen phase may range in amount from about 0.1 to about 20%, preferably from about 0.5 to about 10%, optimally from about 1 to about 5% by weight.

Within the sunscreen phase will be an organic sunscreen agent present in an amount of at least about 25%, preferably from about 25 to about 98%, optimally from about 30 to about 60% by weight of the sunscreen phase.

Amounts of the cosmetic composition relative to the water-insoluble substrate may range from about 20:1 to about 1:20, preferably from about 10:1 to about 1 :10, more preferably from about 2:1 to about 1:2, optimally about 1:1 by weight.

Sunscreen agents of the present invention will have at least one chromophoric group absorbing within the ultraviolet ranging from 290 to 400 nm. Chromophoric organic sunscreen agents may be divided into the following categories (with specific examples) including: p-Aminobenzoic acid, its salts and its derivatives (ethyl, isobutyl, glyceryl esters; p-dimethylaminobenzoic acid); Anthranilates (o-aminobenzoates; methyl, menthyl, phenyl, benzyl, phenylethyl, linalyl, terpinyl, and cyclohexenyl esters); Salicylates (octyl, amyl, phenyl, benzyl, menthyl, glyceryl, and dipropyleneglycol esters); Cinnamic acid derivatives (menthyl and benzyl esters, alpha-phenyl cinnamonitrile; butyl cinnamoyl pyruvate); Dihydroxycinnamic acid derivatives (umbelliferone, methylumbelliferone, methylaceto-umbelliferone); Trihydroxycinnamic acid derivatives (esculetin, methylesculetin, daphnetin, and the glucosides, esculin and daphnin); Hydrocarbons (diphenylbutadiene, stilbene); Dibenzalacetone and benzalacetophenone; Naphtholsulfonates (sodium salts of 2-naphthol-3,6-disulfonic and of 2-naphthol-6,8-disulfonic acids); Dihydroxy-naphthoic acid and its salts; o- and p-Hydroxybiphenyidisulfonates; Coumarin derivatives (7-hydroxy, 7-methyl, 3-phenyl); Diazoles (2-acetyl-3-bromoindazole, phenyl benzoxazole, methyl naphthoxazole, various aryl benzothiazoles); Quinine salts (bisulfate, sulfate, chloride, oleate, and tannate); Quinoline derivatives (8-hydroxyquinoline salts, 2-phenylquinoline); Hydroxy- or methoxy-substituted benzophenones; Uric and vilouric acids; Tannic acid and its derivatives (e.g., hexaethylether); (Butyl carbityl) (6-propyl piperonyl) ether; Hydroquinone; Benzophenones (Oxybenzone, Sulisobenzone, Dioxybenzone, Benzoresorcinol, 2,2′,4,4′-Tetrahydroxybenzophenone, 2,2′-Dihydroxy-4,4′-dimethoxybenzophenone, Octabenzone; 4-Isopropyidibenzoylmethane; Butylmethoxydibenzoylmethane; Etocrylene; and 4-isopropyl-dibenzoylmethane).

Particularly useful are: 2-ethylhexyl p-methoxycinnamate, 4,4′-t-butyl methoxydibenzoylmethane, 2-hydroxy-4-methoxybenzophenone, octyidimethyl p-aminobenzoic acid, digalloyltrioleate, 2,2-dihydroxy-4-methoxybenzophenone, ethyl 4-5 [bis(hydroxypropyl)] aminobenzoate, 2-ethylhexyl-2-cyano-3,3-diphenylacrylate, 2-ethylhexylsalicylate, glyceryl p-aminobenzoate, 3,3,5-trimethylcyclohexylsalicylate, methylanthranilate, p-dimethylaminobenzoic acid or aminobenzoate, 2-ethylhexyl p-dimethylaminobenzoate, 2-phenylbenzimidazole-5-sulfonic acid, 2-(p-dimethylaminophenyl)-5-sulfoniobenzoxazoic acid and mixtures thereof.

Suitable commercially available organic sunscreen agents are those identified under the following table.

TABLE I

| CTFA NAME | TRADE NAME | SUPPLIER |
|---|---|---|
| Benzophenone-3 | UVINUL M-40 | BASF Chemical Co. |
| Benzophenone-4 | UVINUL MS-40 | BASF Chemical Co. |
| Benzophenone-8 | SPECTRA-SORB UV-24 | American Cyanamid |
| DEA-Methoxycinnamate | BERNEL HYDRO | Bernel Chemical |
| Ethyl dihydroxypropyl- | AMERSCREEN P | Amerchol Corp. |
| PABA | | |
| Glyceryl PABA | NIPA G.M.P.A. | Nipa Labs. |
| Homosalate | KEMESTER HMS | Humko Chemical |
| Menthyl anthranilate | SUNAROME UVA | Felton Worldwide |
| Octocrylene | UVINUL N-539 | BASF Chemical Co. |
| Octyl dimethyl PABA | AMERSCOL | Amerchol Corp. |
| Octyl methoxycinnamate | PARSOL MCX | Givaudan Corp. |
| Octyl salicylate | SUNAROME WMO | Felton Worldwide |
| PABA | PABA | National Starch |
| 2-Phenylbenzimidazole- | EUSOLEX 232 | EM Industries |
| 5-sulphonic acid | | |
| TEA salicylate | SUNAROME W | Felton Worldwide |
| 2-(4-Methylbenzylidene)- | EUSOLEX 6300 | EM Industries |
| camphor | | |
| Benzophenone-1 | UVINUL 400 | BASF Chemical Co. |
| Benzophenone-2 | UVINUL D-50 | BASF Chemical Co. |
| Benzophenone-6 | UVINUL D-49 | BASF Chemical Co. |
| Benzophenone-12 | UVINUL 408 | BASF Chemical Co. |
| 4-Isopropyl dibenzoyl | EUSOLEX 8020 | EM Industries |
| methane | | |
| Butyl methoxy dibenzoyl | PARSOL 1789 | Givaudan Corp. |
| methane | | |
| Etocrylene | UVINUL N-35 | BASF Chemical Co. |

Most preferred are organic sunscreens in liquid form when at ambient (25° C.) temperature. Illustrative is octyl methyoxycinnamate.

A surfactant system will also be present in the cosmetic composition. The system must function to stably dispense the sunscreen phase within the water phase. Surfactant system may be incorporated into either of the other two phases. Amounts of the surfactant systems may range from about 0.1 to about 20%, preferably from about 1 to about 10%, optimally from about 2 to about 6% by weight of the composition.

The surfactant may be selected from the group consisting of anionic, nonionic, cationic and amphoteric actives. Particularly preferred nonionic surfactants are those with a C₁₀-C₂₀ fatty alcohol or acid hydrophobe condensed with from about 2 to about 100 moles of ethylene oxide or propylene oxide per mole of hydrophobe; the C₂-C₁₀ alkyl phenols condensed with from 2 to 20 moles of alkylene oxide; mono- and di- fatty acid esters of ethylene glycol; fatty acid monoglyceride; sorbitan, mono- and di- C₈-C₂₀ fatty acids; and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C₈-C₂₀ acyl isethionates and combinations thereof.

Nonionic surfactants are preferred. Particularly effective are combinations of sorbitan esters such as Polysorbate 20 and polyoxyethylene (from 2 to 100 E.O.) fatty alcohols or acids such as PEG 40 hydrogenated castor oil (commercially available as Chremophore® RH-40).

Compositions of the present invention may also contain C₁-C₂₀ alpha- and beta- hydroxy carboxylic acids and salts thereof. The salts are preferably alkaline metal, ammonium and C₁-C₁₂ alkanolammonium salts and mixtures thereof. The term “alpha-hydroxycarboxylic acids” includes not only hydroxy acids but also alpha- ketoacids and related compounds of polymeric forms of hydroxyacid.

Alpha-hydroxyacids are organic carboxylic acids in which one hydroxyl group is attached to the alpha carbon adjacent the carboxy group. The generic structure is as follows:

(Ra)(Rb)C(OH)COOH

where Ra and Rb are H, F, Cl, Br, alkyl, aralkyl or aryl group of saturated or unsaturated, isomeric or non-isomeric, straight or branched chain or cyclic form, having 1 to 25 carbon atoms, and in addition Ra and Rb may carry OH, CHO, COOH and alkoxy groups having 1 to 9 carbon atoms. The alpha-hydroxyacids may be present as a free acid or in lactone form, or in a salt form with an organic base or an inorganic alkali. The alpha-hydroxyacids may exist as stereoisomers as D, L, and DL forms when Ra and Rb are not identical.

Typical alkyl, aralkyl and aryl groups for Ra and Rb include methyl, ethyl, propyl, isopropyl, butyl, pentyl, octyl, lauryl, stearyl, benzyl and phenyl. Most preferred among the alpha-hydroxyacids are glycolic acid, lactic acid, alpha-hydroxycaprylic acid, gluconolactone and combinations thereof.

Among the beta-hydroxycarboxylic acids, the most prominent and useful is salicylic acid.

Amounts of the hydroxy carboxylic acids may range from about 0.01 to about 15%, preferably from about 0.1 to about 12%, more preferably from about 1 to about 8%, optimally from about 2 to about 8% by weight of the total cosmetic composition.

Humectant may be incorporated into compositions of the present invention. Humectants are normally polyols. Representative polyols include glycerin, diglycerin, polyalkylene glycols and more preferably alkylene polyols and their derivatives including propylene glycol, dipropylene glycol, polypropylene glycol, polyethylene glycol and derivatives thereof, sorbitol, hydroxypropyl sorbitol, hexylene glycol, 1,2-butylene glycol, 1,2,6-hexanetriol, isoprene glycol, 2-methyl-1,3-propanediol, ethoxylated glycerol, propoxylated glycerol and mixtures thereof. Amounts of the humectant may range from about 0.01 to about 20%, preferably from about 0.05 to about 5%, optimally from about 0.1 to 2% by weight of the composition.

Small amounts of emollients may be formulated with the sunscreen phase. These emollients may be selected from hydrocarbons, silicones, fatty alcohols, synthetic or natural esters and combinations thereof. Amounts of the emollients may range from about 0.01 to about 10%, preferably from about 0.1 to about 2%, optimally from about 0.3 to about I% by weight of the composition.

Hydrocarbons encompass mineral oil, terpenes (such as squalene) and isoparaffins.

Silicone oils may be divided into the volatile and non-volatile variety. The term “volatile” as used herein refers to those materials which have a measurable vapor pressure at ambient temperature. Volatile -silicone oils are preferably chosen from cyclic or linear polydimethylsiloxanes containing from about 3 to about 9, preferably from about 4 to about 5, silicon atoms. Linear volatile silicone materials generally have viscosities less than about 5 centistokes at 25° C. while cyclic materials typically have viscosities of less than about 10 centistokes. Examples of commercially available volatile silicone oils are Dow Corning® 344 and Dow Corning® 345.

Nonvolatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 100,000 centistokes at 25° C. Among the preferred non-volatile emollients useful in the present compositions are the polydimethyl siloxanes having viscosities from about 10 to about 400 centistokes at 25° C.

Silicone copolyols are useful as both emollient and emulsifying materials within the context of the present invention. Particularly preferred are dimethiconols which may be linear or branched with average number molecular weight ranging from about 1,000 to about 1 million, preferably from about 20,000 to about 500,000, optimally from about 40,000 to about 100,000. Dimethiconols may be formulated as microemulsions in which the silicone is at levels ranging from about 1 to about 95%, preferably from about 10 to about 60%, optimally from about 20 to about 40% by weight of the microemulsion ingredient. Pre-formed microemulsions are available from suppliers such as Dow Corning, General Electric, Union Carbide, Wacker Chemie, Shin Etsu, and Toray Silicone Company. Particularly preferred is a linear dimethiconyl microemulsion at 25% silicone with a maximum particle size of 40 nm, pH 6.5-8 and surfactant combination of dodecylbenzene sulphonic acid triethanolamine/Laureth-24 available from Dow Corning under the trademark DC 2-1870.

Preservatives can desirably be incorporated into the cosmetic compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are phenoxyethanol, methyl paraben, propyl paraben, imidazolidinyl urea, sodium dehydroacetate and benzyl alcohol. The preservatives should be selected having regard for the use of the composition and possible incompatibilities between the preservatives and other ingredients in the composition. Most preferred is iodopropynyl butylcarbamate available from Lonza Corporation under the trademarks Glydant Plus and Glycasil L. Preservatives are preferably employed in amounts ranging from 0.001% to 2% by weight of the composition.

Compositions of the present invention may further include herbal extracts. Illustrative extracts include Centella Asiatica, Ginseng, Ginko Biloba, Chamomile, Green Tea, Scuilcap, Nettle Root, Swertia Japonica, Fennel and Aloe Vera extracts and combinations thereof. Amounts of each of the extracts on an actives basis may range from about 0.00001 to about 1%, preferably from about 0.00 I to about 0.5%, optimally from about 0.005 to about 0.2% by weight of the composition.

Minor adjunct ingredients may also be present in the compositions. Among these may be vitamins such as Vitamin E esters, Vitamin C, Vitamin A esters, Panthenol and any of the Vitamin B complexes. Retinoids may be employed including retinol, retinyl linoleate, retinyl acetate, retinoic acid and combinations thereof. Anti-irritant agents may also be present including those of steviosides, alpha-bisabolol and glycyhrizzinate salts. Each vitamin, retinoid or anti-irritant agent may be present in amounts ranging from about 0.0001 to about 1.0%, preferably from about 0.001 to about 0.5%, optimally from about 0.01 to about 0.3% by weight of the composition.

The impregnating cosmetic compositions can exhibit pH properties ranging from pH 2 to 10. A preferred embodiment has pH being relatively low, for instance, a pH from about 2 to about 6.5, preferably from about 2.5 to about 4.5.

Another important feature of the present invention is that of a substrate which is a water insoluble substance. By “water insoluble” is meant the substrate does not dissolve in or readily break apart upon immersion in water. A wide variety of materials can be used as the substrate. The following nonlimiting characteristics may be desirable: (I) sufficient wet strength for use, (ii) sufficient abrasivity, (iii) sufficient loft and porosity, (iv) sufficient thickness, (v) appropriate size, and (vi) non-reactive with components of the impregnating composition.

Nonlimiting examples of suitable substrates which meet the above criteria include nonwoven substrates, woven substrates, hydroentangled substrates, air entangled substrates and the like. Preferred embodiments employ nonwoven substrates since they are economical and readily available in a variety of materials. By nonwoven is meant that the layer is comprised of fibers which are not woven into a fabric but rather are formed into a sheet, particularly a tissue. The fibers can either be random (i.e., randomly aligned) or they can be carded (i.e. combed to be oriented in primarily one direction). Furthermore, the nonwoven substrate can be composed of a combination of layers of random and carded fibers.

Nonwoven substrates may be comprised of a variety of materials both natural and synthetic. By natural is meant that the materials are derived from plants, animals, insects or byproducts. By synthetic is meant that the materials are obtained primarily from various man-made materials or from material that is usually a fibrous web comprising any of the common synthetic or natural textile-length fibers, or mixtures thereof.

Nonlimiting examples of natural materials useful in the present invention are silk fibers, keratin fibers and cellulosic fibers. Nonlimiting examples of keratin fibers include those selected from the group consisting of wool fibers, camel hair fibers, and the like. Nonlimiting examples of cellulosic fibers include those selected from the group consisting of wood pulp fibers, cotton fibers, hemp fibers, jute fibers, flax fibers, and mixtures thereof. Wood pulp fibers are preferred while all cotton fibers (e.g. cotton pads) are normally avoided.

Nonlimiting examples of synthetic materials useful in the present invention include those selected from the group consisting of acetate fibers, acrylic fibers, cellulose ester fibers, modacrylic fibers, polyamide fibers, polyester fibers, polyolefin fibers, polyvinyl alcohol fibers, rayon fibers and mixtures thereof. Examples of some of these synthetic materials include acrylics such as Acrilan®, Creslan®, and the acrylonitrile-based fiber, Orlon®; cellulose ester fibers such as cellulose acetate, Arnel®, and Acele®; polyamides such as Nylons (e.g., Nylon 6, Nylon 66, Nylon 610 and the like); polyesters such as Fortrel®, Kodel®, and the polyethylene terephthalate fibers, Dacron®; polyolefins such as polypropylene, polyethylene; polyvinyl acetate fibers and mixtures thereof.

Nonwoven substrates made from natural materials consist of webs or sheets most commonly formed on a fine wire screen from a liquid suspension of the fibers.

Substrates made from natural materials useful in the present invention can be obtained from a wide variety of commercial TO sources. Nonlimiting examples of suitable commercially available paper layers useful herein include Airtex®, an embossed airlaid cellulosic layer having a base weight of about 71 gsy, available from James River Corporation, Green Bay, Wis.; and Walkisoft®, an embossed airlaid cellulosic having a base weight of about 75 gsy, available from Walkisoft U.S.A., Mount Holly, N.C.

Nonwoven substrates made from synthetic materials useful in the present invention can also be obtained from a wide variety of commercial sources. Nonlimiting examples of suitable nonwoven layer materials useful herein include HEF 40-047, an apertured hydroentangled material containing about 50% rayon and 50% polyester, and having a basis weight of about 43 grams per square yard (gsy), available from Veratec, Inc., Walpole, Mass.; HEF 140-102, an apertured hydroentangled material containing about 50% rayon and 50% polyester, and having a basis weight of about 56 gsy, available from Veratec, Inc., Walpole, Mass.; Novenet® 149-191, a thermo-bonded grid patterned material containing about 69% rayon, about 25% polypropylene, and about 6% cotton, and having a basis weight of about 100 gsy, available from Veratec, Inc., Walpole, Mass.; HEF Nubtex® 149-801, a nubbed, apertured hydroentangled material, containing about 100% polyester, and having a basis weight of about 70 gsy, available from Veratec, Inc. Walpole, Mass.; Keybak® 951V, a dry formed apertured material, containing about 75% rayon, about 25% acrylic fibers, and having a basis weight of about 43 gsy, available from Chicopee Corporation, New Brunswick, N.J.; Keybak® 1368, an apertured material, containing about 75% rayon, about 5% polyester, and having a basis weight of about 39 gsy, available from Chicopee Corporation, New Brunswick, N.J.; Duralace® 1236, an apertured, hydroentangled material, containing about 100% rayon, and having a basis weight from about 40 gsy to about 115 gsy, available from Chicopee Corporation, New Brunswick, N.J.; Duralace® 5904, an apertured, hydroentangled material, containing about 100% polyester, and having a basis weight from about 40 gsy to about 115 gsy, available from Chicopee Corporation, New Brunswick, N.J.; Sontaro® 8868, a hydroentangled material, containing about 50% cellulose and about 50% polyester, and having a basis weight of about 60 gsy, available from Dupont Chemical Corp.

Most preferred as a towelette for purposes of this invention are non-woven substrates, especially blends of rayon/polyester and ratios of 10:90 to 90:10, preferably ratios of 20:80 to 80:20, optimally 40:60 to 60:40 by weight. A most useful towelette is a 70:30 rayon/polyester non-woven wipe article.

The substrate can be made into a wide variety of shapes and forms. Generally the substrate is in single use towelette form. Advantageously, the towelettes are folded in a Z-shaped formation. They may be interleaved with one another but preferably are not interleaved. The Z fold consists of a center panel flanked by upper and lower wing panels. The upper and lower wing panels are substantially of equal width and substantially half of a width of the center panel. Each towelette is folded medially in a direction orthogonal to that of the Z-shaped formation. Advantageously the size of the towelette may range in length from 10 to 40 cm, preferably from 15 to 30 cm, optimally from 18 to 24 cm. The width of the towelette may range from 8 to 30 cm, preferably from 10 to 25 cm, optimally from 15 to 20 cm.

Anywhere from 5 to 100, preferably from 10 to 50 single towelettes may be stored within a dispensing pouch, preferably a moisture impermeable pouch. During storage and between dispensing, the pouch is resealable, usually via an adhesive strip covering a dispensing opening. Single towelette containing pouches may also be employed.

The substrates of the present invention can comprise two or more layers, each having a different texture and abrasiveness. The differing textures can result from the use of different combinations of materials or from the use of a substrate having a more abrasive side for exfoliation and a softer, absorbent side for gentle cleansing. In addition, separate layers of the substrate can be manufactured to have different colors, thereby helping the user to further distinguish the surfaces.

Except in the operating and comparative examples, or where otherwise explicitly indicated, all numbers in this description indicating amounts of material ought to be understood as modified by the word “about”.

The following examples will more fully illustrate the embodiments of this invention. All parts, percentages and proportions referred to herein and in the appended claims are by weight unless otherwise illustrated.

### EXAMPLE 1

A composition typical of impregnated fluids of the present invention was formulated in the following manner. Table I lists the components of the composition. Phase A (water) was added to Phase B with continuous mixing until uniformity obtained. Phase C was then folded into the mixture. Components of Phase D were, one by one, added into the combined Phase A, B and C. The resultant composition was heated to 45° C. Components of Phase E were mixed together with vigorous agitation for 3-4 minutes, while heating to 45° C. Phase E was then added to the combined Phase A, B, C and D under moderate agitation. The Brookfield LVT Viscosity of the composition was about 5 cps.

An amount of 4 grams of the composition was impregnated into a polyester/rayon towelette (1.8 gram weight; 6 inch by 8 inch size).

TABLE I

| INGREDIENT | WEIGHT % |
|---|---|
| PHASE A | |
| Water | 80.44 |
| PHASE B | |
| Glycolic Acid/Ammonium Glycolate (Neutralized pH 4.0) | 7.69 |
| PHASE C | |
| DC 2-1870 (Dimethicone Microemulsion) | 6.00 |
| PHASE D | |
| Glycerin | 0.01 |
| Sodium Lauroamphoacetate | 2.08 |
| Centella Asiatica Extract | 0.10 |
| Ginseng Extract | 0.10 |
| Green Tea | 0.10 |
| Ginko Biloba Extract | 0.10 |
| Glydant Plus ® Liquid (DMDM Hydantoin + Iodopropynyl | 0.20 |
| butylcarbamate) | |
| PHASE E | |
| Ammonium Glycyrrhizinate | 0.05 |
| Chremophore ® RH-40 (PEG-40 Hydrogenated Castor Oil) | 0.95 |
| Polysorbate 20 | 0.95 |
| Octyl Methoxycinnamate (Parsol ® MCX) | 1.00 |
| Fragrance | 0.15 |
| Vitamin E Acetate | 0.001 |
| Alpha Bisabolol | 0.03 |
| Glycasil L (10% Iodopropynyl Butylcarbamate) | 0.05 |
| Retinol 50C | 0.001 |

### EXAMPLE 2

Towelettes produced in Example 1 were evaluated for their ability to impart sunscreen protection to facial skin. Clinical tests were done in the following manner.

A total of 20 panelists were selected with all completing the study. Average age range was between 20 and 54 years. Male and female panelists were 3 and 17 in number, respectively. All were Caucasian. The panelists were fair-skin individuals with skin types I, II or III as defined in the Federal Register 43:38260 (1978).

The procedure for this study is found in the Federal Register Vol. 43:38264-38267 (1978). One test site area served to determine each subject's Minimal Erythema Dose (MED). This was executed by exposing the back to a series of time incremental UV exposures at 25% intervals. The individual subject's MED is the shortest time of exposure that produces minimally perceptible erythema at 20 to 24 hours post irradiation. The test area is described as the infrascapular area of the back to the right and left of the midline.

A homosalate standard was delivered to the test site through plastic volumetric syringes. Fifteen minutes after application, a series of UV light exposures in 25% increments, calculated from previously determined MED's, bracketing the intended SPF was administered from the solar simulator to sub sites within the treated area. On the actual day of testing another series of exposures similar to the one given on the previous day was administered to an adjacent untreated, unprotected area of the skin to re-determine the MED.

Another adjacent test site was then selected to perform an SPF determination on the test substance. SPF is calculated as MED Protected Skin divided by MED Unprotected Skin.

Test applications with the towelette involved rubbing in a circular motion, uniformly, over the entire site for 10 seconds with moderate pressure.

Results of the tests on the towelette of Example 1 showed an overall mean SPF of 2.72+/−0.45 delivered to the skin.

### EXAMPLE 3

Experiments were conducted to evaluate the effect of viscosity on the resultant SPF value. The evaluation was an in-vitro method. According to the method, vitro skin (N-19 topography available from MS Inc.) was cut into 7×7 cm pieces and placed into a frame. Test samples were dispensed through a micropipettor in the form of at least 100 dots of sample over a 5.5×5.5 cm area. Coverage of the sample was 2 μL/cm². By use of a finger cot, the sample was lightly spread in an even manner over full 5.5×5.5 cm area of the skin. Then it was set aside to dry at least 15 minutes. An SPF 290 Analyzer (Optometrics Group) instrument was used to measure the sun protection factor.

TABLE II

| | | SAMPLE B (%) |
|---|---|---|
| INGREDIENT | SAMPLE A (%) | (CONTROL) |
| Deionized Water | 94.8 | 92.8 |
| Polysorbate 20 | 2.0 | 2.0 |
| Chremaphore ® RH 40 | 2 | 2 |
| Octylmethoxycinnamate | 1 | 1 |
| Glydant Plus ® | 1 | 1 |
| Sepigel 305 ® (Polyacrylamide) | — | 2 |
| PHYSICAL PROPERTIES/ | | |
| PERFORMANCE | | |
| SPF | 3.33 | 2.55 |
| Brookfield LVT Viscosity* | 5 cps | 16,400 cps |

*Spindle 4 at 30 rpm at 25° C.

Table II outlines the formulations and results. It is seen that the lower viscosity composition resulted in the delivery to skin of an SPF of 3.33. A much more viscous control (B) formulation, thickened with a polyacrylamide, resulted in an SPF of 2.55. Thus, a low viscosity formulation achieves improved sunscreen activity.

### EXAMPLES 3-8

A series of compositions are provided under Table III suitable for impregnation into towelettes according to the present invention.

TABLE III

| | EXAMPLES (WEIGHT %) | | | | | |
|---|---|---|---|---|---|---|
| INGREDIENT | 3 | 4 | 5 | 6 | 7 | 8 |
| Phase A | | | | | | |
| Water | 75.00 | 75.00 | 75.00 | 75.00 | 75.00 | 75.00 |
| Phase B | | | | | | |
| Glycolic Acid/Ammonium | 6.00 | — | — | 5.00 | 4.00 | — |
| Glycolate (Neutralized | | | | | | |
| pH 4.0) | | | | | | |
| Potassium Lactate | — | 6.00 | — | — | — | — |
| Lactic Acid | — | — | 6.00 | — | — | 4.00 |
| Phase C | | | | | | |
| DC 2-1870 (Dimethicone | 6.00 | 6.00 | 6.00 | 6.00 | 6.00 | 6.00 |
| Microemulsion) | | | | | | |
| Phase D | | | | | | |
| Glycerin | 0.01 | 0.01 | 0.01 | 0.01 | 0.01 | 0.01 |
| Sodium Lauroamphoacetate | 2.00 | 1.00 | — | — | — | 1.00 |
| Sodium Cocoyl Isethionate | — | 1.00 | 1.00 | — | — | — |
| Sodium Lauryl Sarcosinate | — | — | — | 1.00 | — | — |
| Sodium Cocoamidopropyl | — | — | — | — | 1.00 | 1.00 |
| Betaine | | | | | | |
| Herbal Extracts | 0.40 | 0.40 | 0.40 | 0.40 | 0.40 | 0.40 |
| Glydant Plus Liquid | 0.20 | 0.20 | 0.20 | 0.20 | 0.20 | 0.20 |
| Phase E | | | | | | |
| Ammonium Glycyrrhizinate | 0.05 | 0.05 | 0.05 | 0.05 | 0.05 | 0.05 |
| PEG-40 Hydrogenated | 0.95 | 0.95 | 0.95 | 0.95 | 0.95 | 0.95 |
| Castor Oil | | | | | | |
| Polysorbate 20 | 0.95 | 0.95 | 0.95 | 0.95 | 0.95 | 0.95 |
| Octyl Methoxycinnamate | 2.00 | 1.00 | — | — | — | — |
| Octocrylene | — | — | 1.00 | — | — | — |
| Benzophenone-3 | — | — | — | 1.00 | — | — |
| Octyl Salicylate | — | — | — | — | 1.00 | — |
| Butyl Methoxy Dibenzoyl | — | 1.00 | — | — | — | — |
| Methane | | | | | | |
| Menthyl Anthranilate | — | — | — | — | — | 1.00 |
| Fragrance | 0.15 | 0.15 | 0.15 | 0.15 | 0.15 | 0.15 |
| Vitamin E Acetate | 0.001 | 0.001 | 0.001 | 0.001 | 0.001 | 0.001 |
| Alpha Bisabolol | 0.03 | 0.03 | 0.03 | 0.03 | 0.03 | 0.03 |
| Glycasil L | 0.05 | 0.05 | 0.05 | 0.05 | 0.05 | 0.05 |
| Retinol 50C | 0.001 | 0.001 | 0.001 | 0.001 | 0.001 | 0.001 |
| Water | Bal. | Bal. | Bal. | Bal. | Bal. | Bal. |

Each of the compositions listed in Table III is impregnated into a hydroentangled web of pulp in towelette form. Six grams of each formulation is applied to a towelette (2.2 gram weight; 6 inch by 8 inch size). Towelettes are then stacked, thirty per stack. These are then sealed within a flexible pouch having an opening covered by an adhesively sealed closure flap.

The foregoing description and examples illustrate selected embodiments of the present invention. In light thereof variations and modifications will be suggested to one skilled in the art, all of which are within the spirit and purview of this invention.

What is claimed is:

1. A cosmetic towelette comprising:
(a) a water-insoluble substrate; and
(b) a cosmetic composition impregnated into the substrate, the composition having a viscosity ranging from about 1 to about 10,000 cps, as measured on a Brookfield LVT Viscometer using spindle 4 at 30 rpm at 25° C., the composition comprising:
(i) from about 80 to about 99% of a water phase;
(ii) from about 0.1 to about 20% of a sunscreen phase immiscible with the water phase, the sunscreen phase including at least 25% of an organic sunscreen agent; and
(iii) from about 0.1 to about 10% of a surfactant system to stably disperse the sunscreen phase within the water phase.

2. The towelette according to claim 1 wherein the composition has a viscosity ranging from 5 to 500 cps.

3. The towelette according to claim 1 wherein the sunscreen phase comprises from 25 to 98% organic sunscreen agent.

4. The towelette according to claim 1 wherein the sunscreen phase comprises from 30 to 60% organic sunscreen agent.

5. The towelette according to claim 1 wherein the organic sunscreen is octylmethoxycinnamate.

6. The towelette according to claim 1 wherein the surfactant system comprises a polyoxyethylene hydrogenated castor oil.

7. The towelette according to claim 1 wherein the composition has a pH ranging from about 2 to about 6.5.

8. The towelette according to claim 1 wherein the composition comprises from about 0.01 to about 15% of an alpha- or beta- hydroxycarboxylic acid or salts thereof.

9. A method for imparting sunscreen protection to skin comprising:
(i) providing a cosmetic towelette comprising:
(a) a water-insoluble substrate; and
(b) a cosmetic composition impregnated into the substrate, the cosmetic composition having a viscosity ranging from about 1 to about 10,000 cps, as measured on a Brookfield LVT Viscometer using spindle 4 at 30 rpm at 25° C., the composition comprising:
(i) from about 80 to about 99% of a water phase;
(ii) from about 0.1 to about 20% of a sunscreen phase immiscible with the water phase, the sunscreen phase including at least 25% of an organic sunscreen agent; and
(iii) from about 0.1 to about 10% of a surfactant system to stably disperse the sunscreen phase within the water phase;
(ii) applying the towelette to the skin.
